(19) United States
(12) Patent Application Publication
(10) Pub. No.: US 2011/0003796 A1
(43) Pub. Date: Jan. 6, 2011

(54) METHOD FOR THE TREATMENT OF PSYCHIC DISORDERS

(75) Inventors: Analia Gabriela REINES, Buenos Aires (AR); Carlos Horacio LAINO, La Rioja City (AR)

Correspondence Address: YOUNG & THOMPSON, 209 Madison Street, Suite 500, Alexandria, VA, 22314, US

(73) Assignees: Consejo Nacional de Investigaciones Cientificas y Tecnicas (Conicet), Buenos Aires (AR); Universidad Nacional de la Rioja, La Rioja (AR)

(21) Appl. No.: 12/497,943

(22) Filed: Jul. 6, 2009

(51) Int. Cl. A61K 31/55; A61K 31/20

(52) U.S. Cl. 514/214.02; 514/558; 514/560

(57) ABSTRACT

Method for the treatment of psychiatric disorders, comprising administering to a subject in need thereof an amount of fatty acids and a suboptimal dose of at least one antidepressant. Wherein the fatty acid may be omega-3 (ω3), for example the docohexaenoic acid (DHA) and the eicosapentaenoic acid (EPA). The omega-3 fatty acid may be administered orally, in amounts that may be variable, for example in amounts between 0.15 and 1.00 g/kg/day. The antidepressant may be any antidepressant, preferably the antidepressant is fluoxetine or mirtazapine in sub-optimal doses.

The present invention is related to methods for the treatment of psychic disorders and methods for modifying body weight, comprising administering to a subject in need thereof an amount of omega-3 fatty acids and a suboptimal dose of at least one anti-depressant. Wherein, the omega-3 fatty acid (ω-3) may be, the docohexaenoic acid (DHA) or the eicosapentaenoic acid (EPA). The fatty acid may be administered orally, in amounts that may be variable, for example in amounts between 0.15 and 1.00 g/kg/day. The anti-depressant may be any anti-depressant, preferably the anti-depressant is fluoxetine or mirtazapine in sub-optimal doses.

### BACKGROUND OF THE INVENTION

Of all human body organs (not including fat tissue), the nervous system is the one with the higher content of lipids. The dry weight of an adult brain is 50% to 60% of lipids, and 35% of the contents of lipids are poly-unsaturated fatty acids (PUFAs) (Haag M., Can J Psychiatry 2003. 48(3):195-203). Among the different PUFAs the docosahexaenoic acid (DHA, an omega-3 fatty acid) and the arachidonic acid (AA, an omega-6 fatty acid) are found in a higher concentration. The AA (20:4 n-6) and DHA (22:6 n-3) have important functions in the maintenance of the integrity and fluidity of nervous cell membrane and in the contribution to the neuronal signal transduction. It has been shown that the DHA is especially important in the prenatal development of the brain, where it seems to play an essential role in synaptogenesis (Martin R E, Bazan N G. J Neurochem 1992, 59(1):318-325 and Green P, Glozman S, Kamensky B, et al., J Lipid Res 1999. 40(5):960-966).

The lack of DHA is related to neurophysiologic deficiencies including cognitive impairment (Birch E E, Garfield S, Hoffman D R, et al., Dev Med Child Neurol 2000. 42(3) :174-181), reduced visual acuity (Birch E E, Hoffman D R, Uauy R, et al., Pediatr Res 1998. 44(2):201-209) and reduced cerebellar function (Jamieson E C, Farquharson J, Logan R W, et al., Lipids 1999. 34(10):1065-1071).

In adult mammals, an optimal balance between omega-3 and omega-6 fatty acids is almost essential for normal neuronal functioning. It has been suggested that an imbalance in the omega-3 and omega-6 fatty acids ratio in the diet could be involved in the pathophysiology of different nervous system disorders, particularly psychiatric disorders. This imbalance could be the etiologic mechanism through which some psychiatric disorders are developed in individuals. Therefore, abnormalities in the PUFA metabolism could be treated through the supplementation with said fatty acids. Within the psychiatric disorders, depression and schizophrenia have been most investigated.

In spite of the availability of antidepressant drugs, it is estimated that between 29% and 46% of patients are resistant to treatments, do not show any clinical response or show a partial response to anti-depressant medications. One approach to deal with the treatment-resistant depression is the use of combination therapies or the addition of a booster drug to increase the effects of the primary medication(s). Among natural compounds are omega-3 fatty acids, which have recently been marketed as potential enhancers of antidepressant effects in treatment-resistant depression (Fava M., [Review] J Clin Psychiatry 2001. 62 (Suppl 18):4-1120 and Logan A C., [Review] [117 refs]. Altern Med Rev 2003. 8(4):410-425).

Serotonin (5-HT) and noradrenaline (NA) are involved in the pathogenesis and the recovery of depression. As a consequence, a new generation of antidepressants has been developed, increasing the neurotransmissions of 5-HT and NA. Mirtazapine increases both 5-HT-mediated neurotransmissions as NA-mediated neurotransmissions, and, in a different way, Fluoxetine is a monoamine reuptake inhibitor, specifically a 5-HT reuptake inhibitor.

The patent document U.S. Pat. No. 6,852,870 discloses a method for the treatment of patients with depression through the administration of omega-3 fatty acids. Said fatty acid may be administered in a substantially pure form, as part of a pharmaceutical composition or as part of a big molecule (triacylglycerol).

The patent document WO/1999/029316 discloses an emulsified pre-concentrated pharmaceutical composition, comprising an omega-3 and an active agent which is slightly soluble in water, for example Cyclosporine.

There are also documents about treatment of depression through the administration of antidepressants and fatty acids. In all cases the doses of anti-depressants are doses stated as optimal for the treatment of depressive disorders.

Generally antidepressant drugs are effective and are well tolerated. However, it is still a concern the high number of patients resistant to medication and who, therefore, are not beneficiated with the drug treatment. In fact, more than 70% of patients under treatment with antidepressant drugs are not satisfied, which is put mainly in evidence with the premature interruption thereof (Lin, E H, Von Korff M, Katon W, et al. The role of the primary care physician in patients' adherence to antidepressant therapy. Med Care 1995; 33:67-74 and Zajecka J M. Clinical issue in long term treatment with antidepressants. J Clin Psychiatry, 2000; 61, 20-25). In agreement with this study by Lin et al, 28% of patients stop taking antidepressants by the first month of starting the treatment and 44% by the third month. Some causes have been identified as responsible for said interruption, being the appearance of adverse effects the most frequent one. For example, Fluoxetine can produce sexual impairment (impotence, alterations in ejaculation), anorexia first and the subsequent loss of weight, extrapyramidal adverse effects, cardiovascular alterations (palpitations, arrhythmias), alterations of nervous system (anxiety, nervousness, insomnia, tremors, somnolence, decrease of libido), nausea, vomiting and diarrhea, dryness of the mouth, dyspepsia (Khawam, E. A.; Lauencic, G; Malone, D. A. Side Effects of antidepressant: An overview. Cleveland Clinic Journal of Medicine April 2006 vol. 73 4 351-353). While Mirtazapine may cause somnolence, dizziness, anxiety, confusion, increase of body weight and appetite, dryness of the mouth, constipation, nausea and vomiting, dyspepsia (Khawam, E. A.; Lauencic, G; Malone, D. A. Side Effects of antidepressant: An overview. Cleveland Clinic Journal of Medicine April 2006 vol. 73 4 351-353). Moreover, there are evidences supporting the fact that antidepressant drugs may induce the worsening of depression and a trend to suicide in certain patients during early stages of treatment. Different placebo-controlled studies indicate that antidepressant drugs induce suicidal thoughts and behaviors especially in children, adolescents and young adults (ages between 18 and 24 years old) with major depression as well as with other psychiatric disorders. Thus, it is recommended that any patient who starts a therapy with antidepressants should be appropriately controlled to establish the worsening or changes of behavior as well as a higher trend to suicide.

Forced Swimming Tests (FST) allow to identify, in rodents, treatments with anti-depressant effects in humans (Porsolt R D, Le Pichon M and Jalfre M, Nature. 1977; 266: 730-732 and Detke M J, Rickels M and Lucki, I. Psychopharmacol T121T (1995), pp. 66-72, 29).

### BRIEF DESCRIPTION OF THE INVENTION

The present invention provides a method for the treatment of psychic disorders, comprising administering to a subject in need thereof an amount of omega-3 fatty acids and a suboptimal dose of at least one anti-depressant. Wherein the fatty acid omega-3 (ω-3) may be the docohexaenoic acid (DHA) and the eicosapentaenoic acid (EPA). The omega-3 fatty acid may be administered orally, in amounts that may be variable, for example in amounts between 0.15 and 1.00 g/kg/day. The anti-depressant may be any antidepressant, preferably the antidepressant is fluoxetine or mirtazapine in sub-optimal doses. Said doses may not produce the typical side effects of antidepressants, for example effects such as anxiety, nervousness, nausea, dryness of the mouth, dyspepsia, cardiovascular alterations, alteration of appetite and of body weight, sexual impairment or increasing the trend to suicide, sub-optimal doses may be between ½ and 1/100 of the optimal dose, or doses lower than 5 mg/kg, or doses lower than 3 mg/kg. The anti-depressant may be administered orally or systemically. Any administration route is within the scope of the present invention. The method of the invention may be applied to individuals suffering from any psychiatric disorder, for example major depression, bipolar depression, unipolar depression, depression resistant to drug treatments.

It is also provided a method for modifying, preferably for reducing body weight of an individual, comprising administering to a subject in need thereof an amount of fatty acids and a suboptimal dose of at least one antidepressant. Wherein the fatty acid may be omega-3 (ω-3), for example docohexaenoic acid (DHA) or the eicosapentaenoic acid (EPA). The fatty acid may be administered orally, in amounts that may be variable, for example in amounts between 0.15 and 1.00 g/kg/day. The anti-depressant may be any antidepressant, preferably the antidepressant is mirtazapine in sub-optimal doses. Said doses may not produce typical side effects of anti-depressants, for example effects such as anxiety, nervousness, nausea, dryness of the mouth, dyspepsia, cardiovascular alterations, alteration of appetite and of body weight, sexual impairment or increasing the trend to hoc suicide, sub-optimal doses may be between ½ and 1/100 of the optimal dose, or doses lower than 5 mg/kg, or doses lower than 3 mg/kg. The antidepressant may be administered orally or systemically. Any administration route is within the scope of the present invention.

### DESCRIPTION OF DRAWINGS

FIG. 1 shows the results of immobility and forced swimming tests (FST) in rats treated with omega-3 (0.15; 0.30; 0.72 and 1.0 g/kg/day), bars represent the mean±S.E.M. (n=5-9/dose), data was analyzed with one-way ANOVA, followed by the Dunnett's contrast for comparisons with control group;

FIG. 2 shows the effects of diet supplementation with different doses of omega-3 fatty acids in the open field test, identical doses of drugs were administered in the open field test compared to the ones for FST, values are mean±S.E.M., n=5-10 rats per group, **P<0.05 and data was analyzed with one-way ANOVA, followed by the Dunnett's contrast for comparisons with control group;

FIG. 3 show the effects of fluoxetine (in optimal doses), the omega-3 fatty acids and the combination treatments on behaviors in FST, fluoxetine (10 mg/kg) and/or the omega-3 fatty acids (0.72 g/kg/day) were administered, bars represent the mean of the number of counts over a period of 5 minutes of the test (±S.E.M.). *P<0.05; **P<0.01, 7-12 rats per group were tested, c-sal means control diet-saline, c-flx 10 means control diet-fluoxetine 10, ω-3-sal means omega-3 diet-saline, ω-3-fluoxetine 10 means omega-3 diet-fluoxetine 10. Data was analyzed with two-way ANOVA, followed by the Turkey's contrast for multiple comparisons;

FIG. 4 shows the effects of mirtazapine, the omega-3 fatty acids, and the combination treatments in FST tests, mirtazapine in optimal doses (20 mg/kg) and/or the omega-3 fatty acids in dose of 0.72 g/kg/day were administered, bars represent the mean of the number of counts over a period of 5 minutes of the test (±S.E.M.), *P<0.05; **P<0.01; 6-12 rats per group were tested, c-sal means control diet-saline, c-mtz means control diet-mirtazapine 20, ω-3-sal means omega-3 diet-saline, ω-3-mtz 20 means omega-3 diet-mirtazapine 20. Data was analyzed with two-way ANOVA, followed by the Turkey's contrast for multiple comparisons;

FIG. 5 shows the effects of diet supplementation with omega-3 fatty acids (0.72 g/kg/day) and/or fluoxetine (10 mg/kg) in the open field test, fluoxetine in optimal doses (10 mg/kg) and/or the omega-3 fatty acids in dose of 0.72 g/kg/day were administered, bars represent the mean of the number of counts over a period of 5 minutes of the test (±S.E.M.), 7-12 rats per group were tested and data was analyzed with two-way ANOVA;

FIG. 6 shows the effects of diet supplementation with omega-3 fatty acids (0.72 g/kg/day) and/or mirtazapine (20 mg/kg, optimal dose) in the open field test, values are expressed as mean±SEM, 5-7 rats were tested and data was analyzed with two-way ANOVA;

FIG. 7 shows the behavioral effects produced in the FST by fluoxetine in sub-optimal doses (1 mg/kg), omega-3 fatty acids in dose of 0.72 g/kg/day and the combination treatments, bars represent the mean of the number of counts over a period of 5 minutes of the test (±S.E.M.). *P<0.05; **P<0.01; 7-12 rats per group were tested, c-sal means control diet-saline, c-flx means control diet-fluoxetine 1, ω-3-sal means omega-3 diet-saline, ω-3-fluoxetine 1 means omega-3 diet-fluoxetine 1, data was analyzed with two-way ANOVA, followed by the Tuckey's contrast for comparisons with control group;

FIG. 8 shows the behavioral effects produced in the FST by mirtazapine in sub-optimal doses (1 mg/kg), omega-3 fatty acids in dose of 0.72 g/kg/day and the combination treatments, bars represent the mean of the number of counts over a period of 5 minutes of the test (±S.E.M.), *P<0.05; **P<0.01; 6-12 rats per group were tested, c-sal means control diet-saline, c-mtz 1 means control diet-mirtazapine 1, ω-3-sal means omega-3 diet-saline, ω-3-mtz 1 means omega-3 diet-mirtazapine 1, data was analyzed with two-way ANOVA, followed by the Tuckey's contrast for comparisons with control group;

FIG. 9 shows the effects of treatments with fluoxetine in sub-optimal doses (1 mg/kg), the omega-3 fatty acids in dose of 0.72 g/kg/day and the combination treatments in locomotor activity in rats, bars represent the mean of the number of counts over a period of 5 minutes of the test (±S.E.M.), 5-6 rats per group were tested and data was analyzed with two-way ANOVA;

FIG. 10 shows the effects of treatments with mirtazapine in sub-optimal doses (1 mg/kg), the omega-3 fatty acids in dose of 0.72 g/kg/day and the combination treatments in locomotor activity in rats, bars represent the mean of the number of counts over a period of 5 minutes of the test (±S.E.M.), 5-6 rats per group were tested and data was analyzed with two-way ANOVA, followed by the Turkey's contrast for comparisons with control group;

FIG. 11 shows the weight gain of rats after the supplementation with different doses of omega-3 fatty acids, the results are expressed as media±S.E.M, 5-17 rats per group were tested and data was analyzed with two-way ANOVA;

FIG. 12 shows the effects of fluoxetine (10 mg/kg), the omega-3 fatty acids (0.72 g/kg/day) and the combination treatments in the weight gain calculated as the difference between weights at the beginning of the protocol and at the moment of FST (the re-test), the results are expressed as media±S.E.M. **P<0.001; 5-9 rats per group were tested and data was analyzed with two-way ANOVA, followed by the Turkey's contrast for comparisons with control group;

FIG. 13 shows the effects of fluoxetine in sub-optimal doses (1 mg/kg), the omega-3 fatty acids in dose of 0.72 g/kg/day and the combination treatments in the weight gain calculated as the difference between weights at the beginning of the protocol and at the moment of the re-test, the results are expressed as media±S.E.M. **P<0.001, 5 rats per group were tested and data was analyzed with two-way ANOVA, followed by the Turkey's contrast for comparisons with control group;

FIG. 14 shows the effects of mirtazapine (20 mg/kg), the omega-3 fatty acids (0.72 g/kg/day) and the combination treatments in the weight gain calculated as the difference between weights at the beginning of the protocol and at the moment of the re-test, the results are expressed as media±S.E.M. **P<0.001, 10-13 rats per group were tested and data was analyzed with two-way ANOVA, followed by the Turkey's contrast for multiple comparison;

FIG. 15 shows the effects of mirtazapine in sub-optimal doses (1 mg/kg), the omega-3 fatty acids in dose of 0.72 g/kg/day and the combination treatments in the weight gain calculated as the difference between weights at the beginning of the protocol and at the moment of FST (the re-test), the results are expressed as media±S.E.M. **P<0.001; 5-6 rats per group were tested and data was analyzed with two-way ANOVA, followed by the Turkey's contrast for multiple comparison;

FIG. 16 shows the effects of fluoxetine (10 mg/kg), the omega-3 fatty acids (0.72 g/kg/day) and the combination treatments on the content of phospholipids in the brain cortex, the results are expressed as media±S.E.M for individual phospholipids, Abbreviations: PC: phosphatidylcholine, PE: phosphatidylethanolamine, SM: sphingomyelin, PI: phosphatidylinositol and PS: phosphatidylserine, data was analyzed with two-way ANOVA;

FIG. 17 shows the effects of fluoxetine (10 mg/kg), the omega-3 fatty acids (0.72 g/kg/day) and the combination treatments on the content of phospholipids in the hippocampus, the results are expressed as media±S.E.M for individual phospholipids, Abbreviations: PC: phosphatidylcholine, PE: phosphatidylethanolamine, SM: sphingomyelin, PI: phosphatidylinositol and PS: phosphatidylserine, data was analyzed with two-way ANOVA;

FIG. 18 shows the experimental design: the forced swimming and/or open field tests were carried out in the different groups of rats on days 1 and 17 after 16 days of treatment with saline or antidepressants while receiving a control diet or supplemented with omega-3, FST: forced swimming test, OFT: open field test, Diet: control or omega-3 fatty acids, Drugs: fluoxetine or mirtazapine.

FIG. 19 shows the behavioral effects produced in the FST by fluoxetine in sub-optimal doses (0.1 and 0.5 mg/kg), omega-3 fatty acids in dose of 0.72 g/kg/day and the combination treatments, bars represent the mean of the number of counts over a period of 5 minutes of the test (±S.E.M.), *P<0.05; **P<0.01; 5 rats per group were tested, c-sal means control diet-saline, c-flx 0.1 means control diet-fluoxetine 0.1, c-flx 0.5 means control diet-fluoxetine 0.5, ω-3-sal means omega-3 diet-saline, ω-3-flx 0.1 means omega-3 diet-fluoxetine 0.1, ω-3-flx 0.5 means omega-3 diet-fluoxetine 0.5 diet, data was analyzed with two-way ANOVA, followed by the Tuckey's contrast for comparisons with control group.

### DETAILED DESCRIPTION OF THE INVENTION

The object of the present application is to provide a method for the treatment of nervous system disorders, more particularly psychiatric disorders and more particularly depressions. It is shown, for example the anti-depressant effect of the omega-3 fatty acid in combination with sub-optimal doses of anti-depressants, using the forced swimming test (FST).

For example, antidepressants such as fluoxetine or mirtazapine were used. Specifically, the behavioral effects of the omega-3 fatty acids, fluoxetine and mirtazapine were tested in rats using the FST. It is evident that the use of any psychiatric or anti-depressant drug is within the scope of the present invention.

The administration of the omega-3 fatty acids by diet supplementation was carried out during 16 days. The effects of chronic treatments were analyzed with fluoxetine and mirtazapine in rats kept with an omega-3 fatty acid rich diet to determine if the effects of these treatments may give a new pharmacological approach. Also, it was analyzed if these new treatments with effects of antidepressant type on the FST have non-specific effects on locomotor activity.

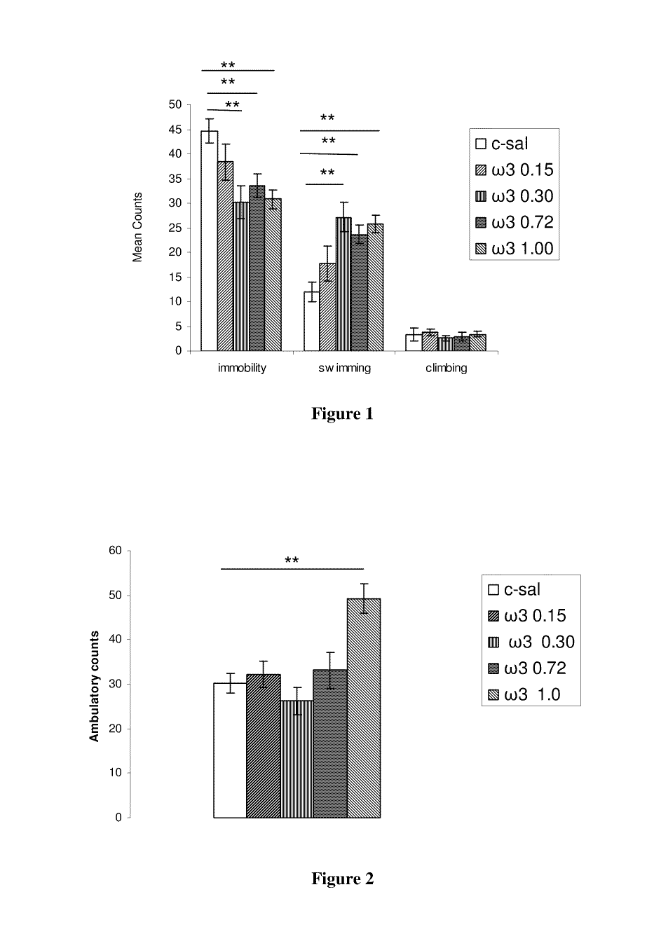

In order to further explore the omega-3 fatty acids antidepressant action and to determine their pharmacological nature, the effects of the omega-3 fatty acids were tested on the FST. The diet supplementation with the omega-3 fatty acids showed dose-related effects on the FST compared with the controls. The omega-3 (0.30; 0.72 and 1.00 g/kg/day) produced a dose-dependent decrease in immobility [F(4, 30)=4.46; p<0.05] and a dose-dependent increase in the corresponding swimming [F(4, 30)=6.79; p<0.001] without significantly affecting the frequency of climbing in rats after 16 days [F(4, 30)=1.24; p=NS] (FIG. 1).

The effects of different doses of omega-3 fatty acids (0.15; 0.30; 0.72 and 1.00 g/kg/day) in the locomotor activity in the open field test are shown in FIG. 2. The omega-3 fatty acids did not change the locomotor activity in doses of 0.15 to 0.72 g/kg/day. However, the locomotor activity increased with doses of 1.00 g/kg/day of omega-3 [F(4, 32)=4.74; P<0.005].

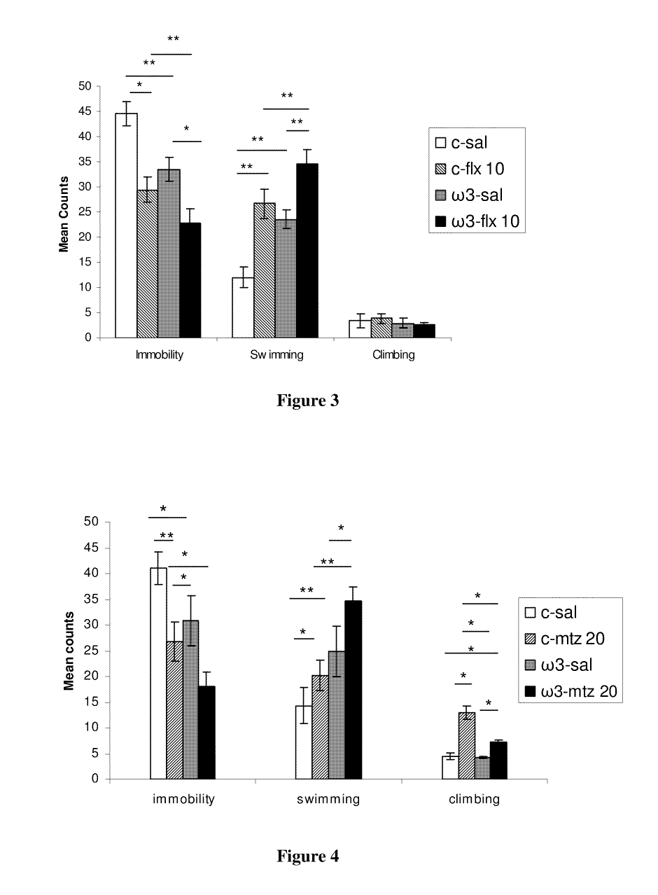

The behavioral effects produced in the FST were evaluated by the administration of fluoxetine (FLX) (10 mg/kg, ip) and/or the diet supplementation with omega-3 fatty acids (0.72 g/kg). The FLX reduces immobility [F(1, 37)=20.91; p<0.0001] and increases swimming [F(1, 37)=24.30; p<0.0001], without modifying the behavioral levels of climbing [F(1, 37)=0.01; p=NS], a behavioral pattern consistent with a serotoninergic mechanism of action. As shown in FIG. 3, omega-3 reduces immobility [F(1, 37)=9.83; p<0.001], and increases the swimming [F(1, 37)=13.93; p<0.001], without affecting the climbing [F(1, 37)=0.90; p=NS], a behavioral profile similar to the one observed with FLX. The combination treatment of FLX/omega-3 fatty acids also reduced the immobility (P<0.01; Tukey) and increased the swimming (P<0.01; Tukey). The effect of the combination of drug and diet was significantly stronger when compared with the effects obtained after the individual administration of FLX or omega-3 (P<0.01 versus FLX and omega-3) producing an additive effect (FIG. 3).

The administration of the antidepressant mirtazapine, which increases the transmissions of 5-HT and NA, produced a behavioral pattern different to the one produced by fluoxetine. The mirtazapine (20 mg/kg, ip) significantly reduced the immobility [F(1, 26)=12, 91; P<0.005] with a corresponding increase in the swimming [F(1, 26)=4.54; P<0.005] and climbing [F(1, 26)=80.20; P<0.005]. The enrichment of diet with omega-3 fatty acids (0.72 g/kg) reduced the immobility [F(1, 26)=6.38; P<0.05] and increased the swimming [F(1, 26)=11.60; P<0.005] in comparison with control group. The combination treatment of MTZ/omega-3 fatty acids reduced the immobility (P<0.01; Tukey) and increased the swimming and climbing (P<0.01; Tukey). The effect of the combination of drug and diet was significantly stronger when compared with the effects obtained after the individual administration of MTZ or omega-3 (P<0.01 versus MTZ and omega-3) producing an additive effect (FIG. 4).

The effects of fluoxetine (10 mg/kg) and/or omega-3 fatty acids were analyzed (0.72 g/kg) in the spontaneous locomotor activity in rats. None of these treatments with antidepressant effects in the FST affected the activity levels when rats were tested in the open field test, instead of the forced swimming test (FIG. 5).

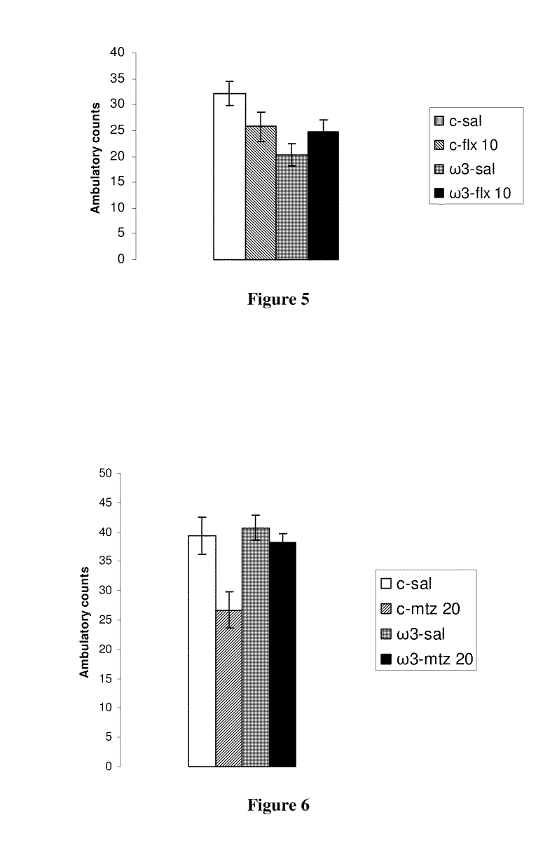

The effects of diet supplementation with omega-3 fatty acids (0.72 g/kg) and/or mirtazapine (20 mg/kg) were tested in the spontaneous locomotor activity in rats. The mirtazapine and the omega-3 fatty acids, alone or in combination, did not interfere with the responses of locomotor activity (p>0.05) (FIG. 6).

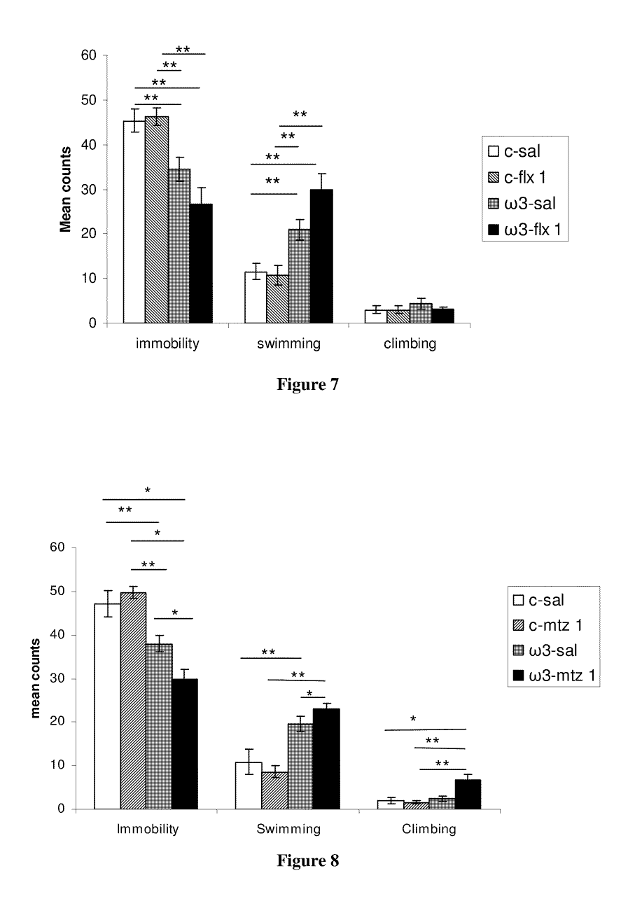

The effects of fluoxetine in a sub-optimal dose (1 mg/kg, ip) and/or omega-3 fatty acids (0.72 g/kg) were tested on behaviors in FST. The two-factor ANOVA revealed that in agreement with the initial results, the omega-3 fatty acids induce a significant reduction of the immobility [F(1, 15)=30.75; p<0.005] and increase the swimming time [F(1, 15)=28.74; p<0.005] (FIG. 7), with no effect of treatment with FLX [F(1, 15)=0.534; P>0.05]. The co-administration of FLX (1 mg/kg i.p.) and omega-3 fatty acids significantly reduced the time of immobility ([F(1, 17)=43.61; P>0.0001]) [P>0.01] and increased the swimming [P>0.05]. There was also a significant interaction between diet and the treatment ([F(1, 15)=3.46; P<0.05]) [P<0.05], indicating that FLX potentiates the antidepressant effect of the omega-3 fatty acids (FIG. 7).

Additional experiments were carried out with a combination of sub-optimal doses of mirtazapine (1 mg/kg, i.p). This dose of mirtazapine did not significantly change the time of immobility compared with control group (p>0.05). Instead, the co-administration of mirtazapine (1 mg/kg, i.p.) and omega-3 fatty acids significantly reduced the time of immobility [F(1, 20)=43.61, P<0.005] and increased the swimming [F(1, 20)=39,1 P<0.005] and the climbing [F(1, 20)=11.1, P<0.005] compared with control animals ([F(1, 17)=43.61; P>0.0001]) ; an interaction was observed between the treatment and the diet (p<0.05), indicating that mirtazapine potentiates the antidepressant effect of the omega-3 fatty acids (FIG. 8).

The ineffective doses of fluoxetine and mirtazapine potentiate the antidepressant effect of the omega-3 fatty acids (FIGS. 7 and 8).

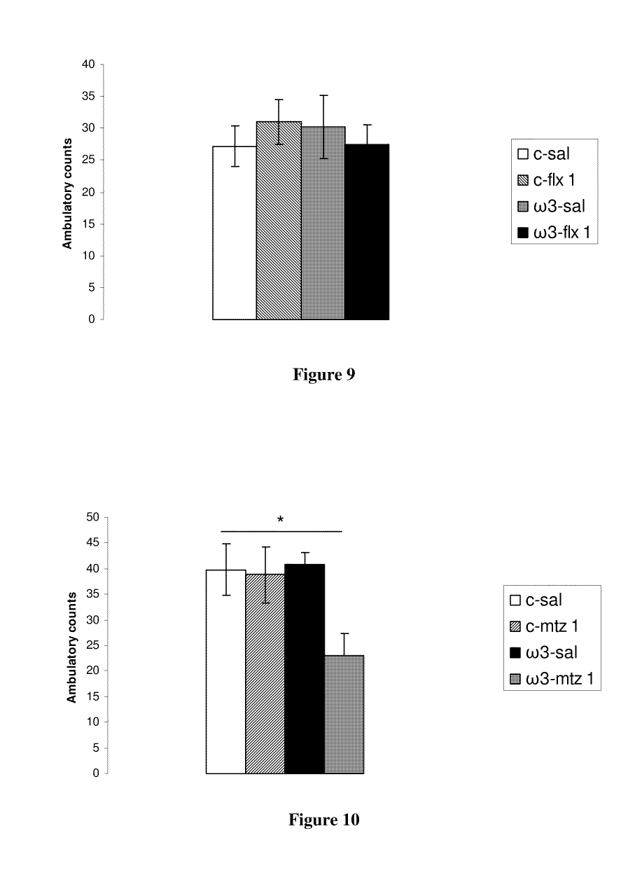

The effects of fluoxetine (1 mg/kg) and/or omega-3 fatty acids were tested (0.72 g/kg) on the spontaneous locomotor activity in rats. None of these treatments with antidepressant effects in the FST affected the levels of activity when rats were tested in the open field chamber, instead of the cylinders of forced swimming during the re-test (FIG. 9).

The effects of diet supplementation with omega-3 fatty acids (0.72 g/kg/day) and/or mirtazapine (1 mg/kg) were tested on the spontaneous locomotor activity in rats. Even though the mirtazapine and omega-3 fatty acids alone did not interfere with the locomotor activity, the combination treatment diminished this behavioral parameter (FIG. 10).

Tests were performed to analyze the body weight gain during chronic treatments with antidepressants and/or omega-3 fatty acids.

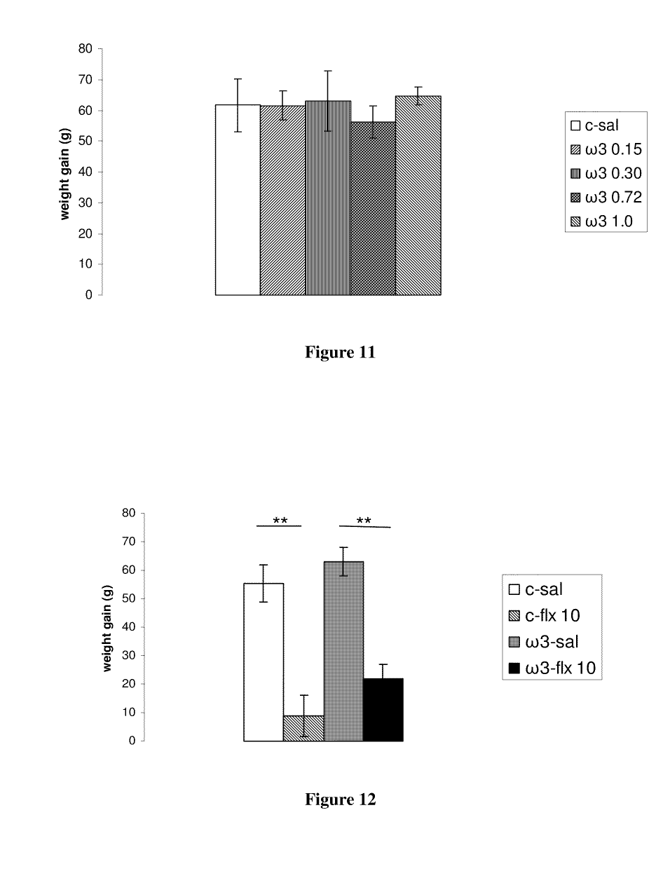

The body weight gain was evaluated with increasing doses of omega-3 fatty acids (0.15-1.00 g/kg/day). In none of the test groups, the supplementation of the diet with omega-3 induced changes in body weight gain (FIG. 11).

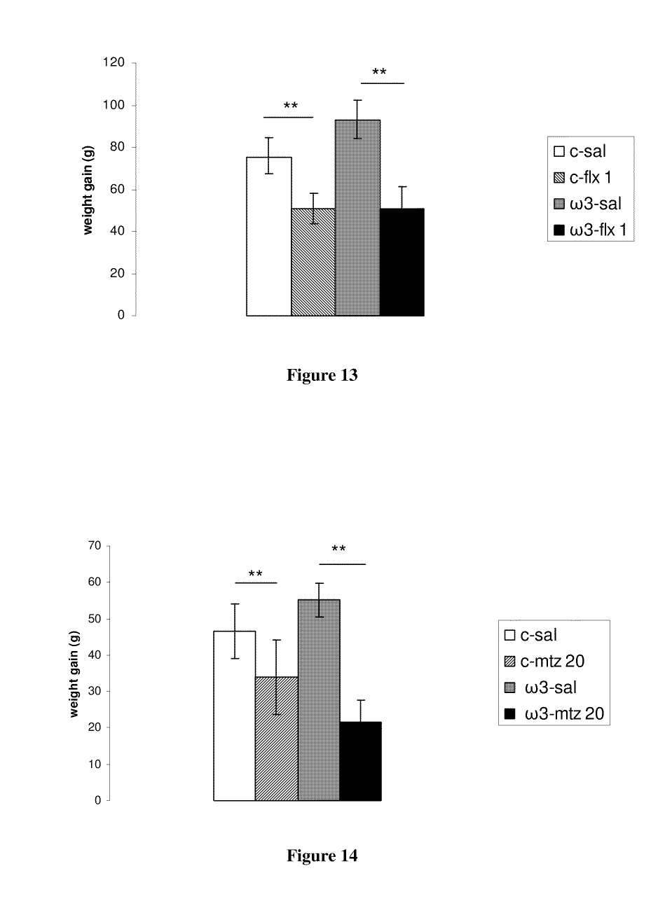

Body weight was evaluated in all of the four groups before treatments with fluoxetine (10 mg/kg) and/or omega-3 fatty acids (0.72 g/kg), and after said treatments. Body weight was significantly lower in animals treated with 10 mg/kg of fluoxetine alone or in combination with omega-3 fatty acids compared with control group (FIG. 12). Body weight gain was reduced although in a lesser extend when sub-optimal doses of FLX were used (1 mg/kg) (FIG. 13).

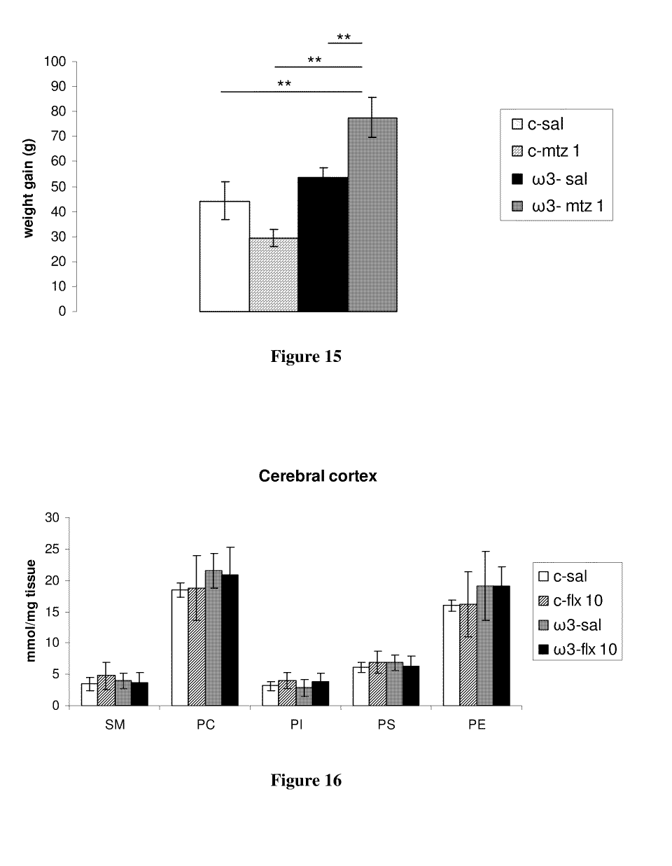

Both the chronic treatment with mirtazapine (20 mg/kg) alone and in combination with omega-3 fatty acids (0.72 g/kg) significantly reduced the body weight gain (FIG. 14). Different results were obtained with the lowest dose of mirtazapine. Mirtazapine alone in sub-optimal doses (1 mg/kg) did not produce any effect, the combination treatment with omega-3 fatty acids (0.72 g/kg) induced an increase in the body weight gain (FIG. 15).

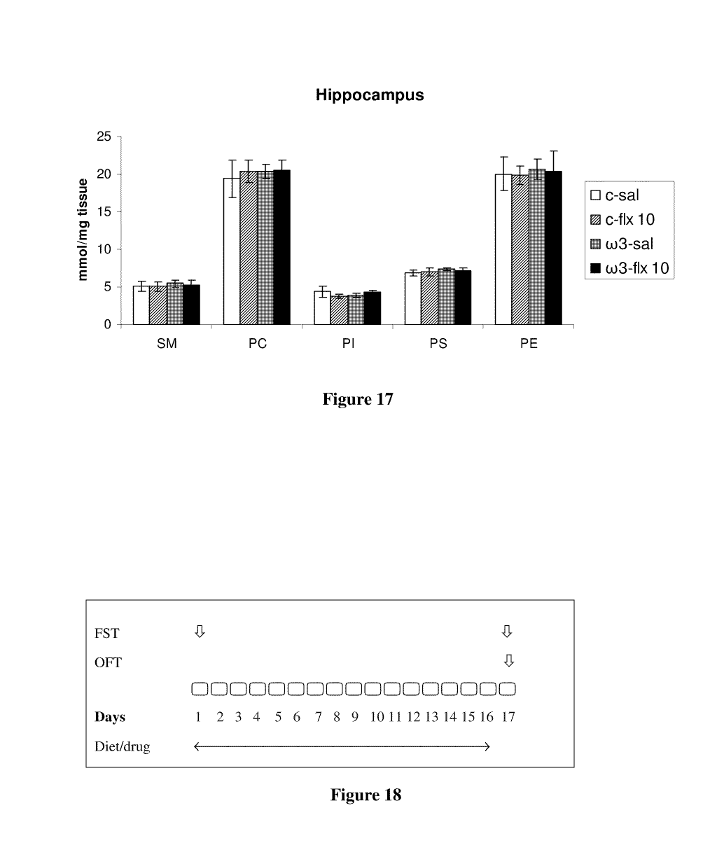

The effects of fluoxetine (10 mg/kg) and/or omega-3 fatty acids (0.72 g/kg) were tested in the phospholipids in the brain cortex (FIG. 16) and in the hippocampus (FIG. 17). We observe that fluoxetine and/or the omega-3 fatty acids did not induce any important change on the classes of phospholipids in the brain cortex and in the hippocampus. None of these treatments with antidepressant effects in the FST affected the content of phospholipids in the hippocampus and the brain cortex, for example as regards phosphatidylcholine, phosphatidylethanolamine, sphingomyelin, phosphatidylinositol and phosphatidylserine.

The present invention shows the pharmacological properties and the benefits of the combined administration of antidepressant drugs in sub-optimal doses and fatty acids, particularly omega-3 fatty acids. It has been reported that chronic stress is a factor which predisposes to depression. In this study, we used the forced swimming test (FST), a well accepted model for proving the anti-depressive action of agents using the stimulus of the forced swimming as a stress condition to generate a behavior characterized by the increase of the time of immobility. It has been described that the appearance of behavioral changes on the FST is due to changes on neurotransmitters and cell signaling pathways in the brain.

The FST implies a design in which the pre-test is carried out first, then the chronic treatment of drug is administered, and after 24 hr the final test (re-test) is shown. This scheme is due to the fact that some anti-depressants may have anxiolytic effects and anxiolytic drugs given before the stressor in the pre-test may reduce the impact of the stressor stimulus on the animal behavior. On the other hand, the administration of anti-depressants after carrying out the stressing activity in the pre-test allows for the evaluation of the action of the drug after the behavioral failure has been developed.

These results show that the omega-3 fatty acids posses antidepressant effects after 16 days of treatment and once the stressor stimulus has been applied. Moreover, the reduction of the duration of the immobility or the increase of the behavior of swimming induced in the FST by this treatment may be proposed as specific, because they are not attributable to changes on the locomotor activity.

The supplementation with omega-3 fatty acids and their combination with sub-optimal doses of fluoxetine or mirtazapine have an antidepressant effect significantly higher than the omega-3 fatty acids alone, suggesting that the antidepressant effects of the omega-3 fatty acids may be potentiated with sub-optimal doses of anti-depressant drugs.

The method of the present invention could be applied to different types of neurological and/or psychic disorders, wherein the disorder has a relationship with the depletion of DHA and EPA, for example in Alzheimer's disease, in heart diseases and depression and others.

Mirtazapine, one widely used antidepressant, induce adverse metabolic effects such an increase of body weight. Surprisingly, this invention shows that mirtazapine (20 mg/kg, ip) produced a significant decrease in body weight, both in rats kept with control diet and with a diet supplemented with omega-3. It is also shown that the chronic administration of one sub-optimal dose of mirtazapine (1 mg/kg) does not interfere with the weight gain as regards rats kept with the control diet. On the other side, the co-administration of mirtazapine in sub-optimal doses (1 mg/kg i.p.) and omega-3 fatty acids significantly increased the weight gain.

The present results demonstrate that the combination treatment of sub-optimal doses of fluoxetine or mirtazapine and omega-3 fatty acids allow to reduce doses of antidepressants to be used, thus reducing the side and adverse effects of antidepressant drugs. The method is also useful to be administered to subjects who need to reduce or increase their body weight.

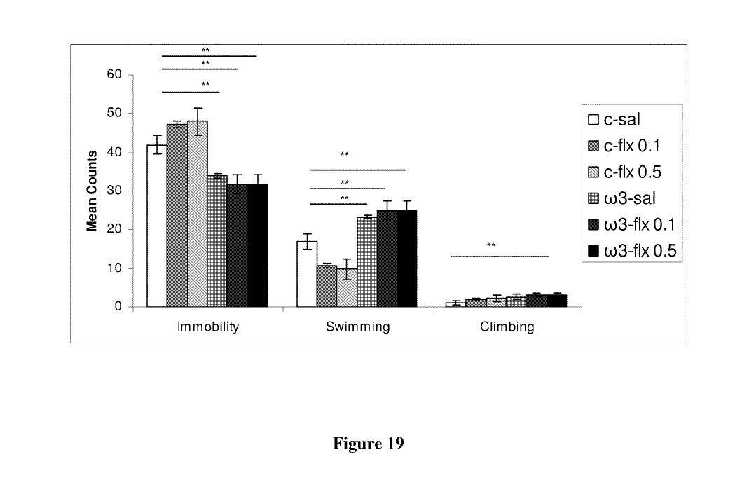

Also the effects of fluoxetine with other sub-optimal doses lower than 1 mg/kg, ip (0.1 and 0.5 mg/kg) and/or omega-3 fatty acids (0.72 g/kg) were tested on behaviors in FST. The two-factor ANOVA revealed, in coincidence with the former results, that the omega-3 fatty acids induce a significant reduction of the immobility [F(1, 20)=40.78; p<0.0001] and increase the swimming time [F(1, 20)=42.86; p<0.0001] (FIG. 19), while the treatments with the sub-optimal doses of FLX lower than 1 mg/kg did not have any effect on the FST [F(2, 20)=0.75; P>0.05]. The co-administration of FLX (0.1 or 0.5 mg/kg i.p.) and omega-3 fatty acids significantly reduced the time of immobility ([F(1, 17)=43.61; P>0.0001]) [P>0.001] and increased the swimming [P>0.001] (FIG. 19), but in no case this effect was higher than the one produced by the individual treatment with omega-3 fatty acids, which indicates that at these doses the FLX did not potentiate the anti-depressant effects of said fatty acids.

This invention is better illustrated according to the following examples, which should not be interpreted as a limitation imposed to the scope thereof. On the contrary, it should be clearly understood that other embodiments, modifications and equivalents may be used and after reading the present specification, it may be suggestive to those skilled in the art without departing from the spirit of the present invention and/or the scope of attached claims.

### EXAMPLES

### Example 1

### Schemes of Treatment

The experiments were carried out with male Wistar rats with a body weight of 200-386 g. Rats were kept in a cycle of 12 hours of light (8:00 a 20:00) and 12 hours of darkness with free access to water and food except during the tests. The rats were divided in four groups, housed in individual polyethylene cages of with three or four rats each (55×38×30 cm) and fed with standard diet for the controls (C) or diet supplemented with omega-3 fatty acids (ω3). The animals were used only once in each test. All tests were performed in agreement with the Guidelines for the Care and Use of Laboratory Animals provided by National Institutes of Health of United States of America.

The Fluoxetine Hydrochloride (FLX) and mirtazapine (MTZ) were administered intra-peritoneally (IP) in a volume equivalent to 1 cc/kg and were freshly prepared each morning. The FLX was dissolved in distilled water and MZT was solubilized in isotonic saline (0.9% NaCl) plus three or four drops of glacial acetic acid to dissolve this solution. The solution of glacial acetic acid in such a little amount does not induce effects per se on the FST or on the locomotive activity (Rénéric, J P. Manuel Bouvardb and Luis Stinus. In the rat forced swimming test, chronic but not subacute administration of dual 5-HT/NA antidepressant treatments may produce greater effects than selective drugs. Behavioural Brain Research, Volume 136, Issue 2, 15 Nov. 2002, Pages 521-532). All control rats were given injections of saline.

The doses of FLX (10 mg/kg) and MTZ (20 mg/kg) produce a more robust effect on the FST, in prior studies and under similar test conditions (Rénéric, J P. and Lucki, I. Antidepressant behavioral effects by dual inhibition of monoamine reuptake in the rat forced swimming test. Psychopharmacology (Berl) 136 (1998), pp. 190-197, and Egawa, T.; Ichimaru, Y.; Imanishi. T and Sawa A. Neither the 5-HT1A- nor the 5-HT2-receptor subtype mediates the effects of fluvoxamine, a selective serotonin reuptake inhibitor, on forced-swimming-induced immobility in mice. Jpn. J. Pharmacol. 68 (1995), pp. 71-75).

The doses of FLX (1 mg/kg) or MTZ (1 mg/kg) not showing an antidepressant effect were selected in agreement with prior studies (Nowakowska E, Chodera A, Kus K. Behavioral and memory improving effects of mirtazapine in rats. Pol J Pharmacol. 1999. 51, 463-469 and Contreras, C. M., The lowest effective dose of fluoxetine in the forced swim test significantly affects the firing rate of lateral septal nucleus neurons in the rat. J. Psychopharmacology 2001, 15, 231-236).

In all cases antidepressant drugs or the saline solution were given once a day for 16 days, the final injection was given 24 hours before the test session (FIGS. 6 and 18).

Four groups of rats were tested. Two groups were fed with 20% casein supplemented with fish oil and the remaining two groups were kept with normal 20% casein.

The omega-3 fatty acids were given as diet supplement in the food enriched with salmon oil for 16 days. Each 1000 mg of highly concentrated salmon oil contains about 30% of omega-3, from which 17% is EPA and 13% is DHA; therefore there were 40 mg of omega-3 fatty acids per gram of enriched food. Rats of the ω3 group received a dose of about 0.72 g/kg/day of omega-3 fatty acids.

The omega-3 fatty acids were supplemented every morning. Control animals were fed with standard diet (20% casein).

The experiments in which low doses of omega-3 were used, said fatty acid was within a range of 0.15 at 1 g/kg/day. The diets were equivalent in the total content of fat, protein, carbohydrates and calories

TABLE 1
Composition of the diet

| | |
|---|---|
| Control | ω3 Diet |

g/kg Diet

| | | |
|---|---|---|
| Calcium Caseinate | 200 | 200 |
| Corn oil | 50 | 50 |
| Choline hydrochloride | 1.5 | 1.5 |
| Mix of vitamins¹ | 10 | 10 |
| Mix of minerals² | 35 | 35 |
| Maltodextrin | 696.9 | 696.9 |
| Salmon oil | — | 11.93 |

¹Composision of supplement of vitamin triturated in sucrose (g/kg of Diet): calcium D-pantotenate, 1.60; nicotinic acid, 3.00; D-biotin, 0.02; menadione, 0.029; thiamine HCl, 0.60; riboflavin, 0.60; folic acid, 0.20; dl-alpha-tocopherol acetate (500 u/g), 15.00; retynil palmitate, (400 uI/g), 0.228; pyridoxine HCl, 0.70; 0.1% cyanocobalamine (triturated in mannitol 1:1000), 2.50; cholecalcipherol, (250000 U/g), 0.40; sucrose, 975.123.
²Composición (g/kg of Diet) as follows: K₂HPO₄, 322.5; CaCO₃, 357; NaCl, 74; MgO, 0.8; MgSO₄ 7H₂O, 146,9; ZnSO₄ 5 H₂O, 0.63; (NH₄)₆Mo₇O₂₄ 4H₂O, 0.008; KI, 0.0078; Na₂SeO₃ 5H₂O, 0.1025; iron and ammonium citrate, 6.06; ZnCl, 1.79; sucrose 91.

TABLE 2
Composition of fatty acids (FA) of test
diets^{a}

Test Diet

| Fatty Acids | CAS | ω3 Diet |
|---|---|---|
| Myristic | 0.11 | 0.76 |
| Palmitoleic | 0.16 | 9.74 |
| C 16:0 palmitic | 6.75 | 16.25 |
| C 18:0 stearic | 3.04 | 4.10 |
| C 18:1 ω9 oleic | 29.2 | 13.05 |
| C 18:2 ω6 linoleic | 58.8 | 1.67 |
| C 18:3 ω3 alpha-linolenic | 0.11 | 0.69 |
| C 20:4 ω6 arachidonic | 1.24 | 1.15 |
| C 20:5 ω3 eicosapentanoic | NC | 17.24 |
| C 22:6 ω3 docosahexaenoic | NC | 12.21 |

^{a}The values are the composition of FA as a percentage of the total of Diet FA, determined by gas chromatography.

### Example 2

### Tests of Response of Rats and Biochemical Assessments

Forced Swimming Test (FST)

The proceeding used was very similar to the one described by Porsolt et al (1978) (Porsolt R D, Anton G, Blavet N, Jalfre M (1978): Behavioural despair in rats: a new model sensitive to antidepressant treatments. Eur J Pharmacol 47:379-391T), except that the water had a depth of 30 cm and the pre-test was performed 17 days before the test session.

The swimming sessions were carried out placing the rats in Plexiglas cylinders (46 cm of height and 20 cm of diameter) which had been previously filled with water (23-25° C.) to 30 cm from the bottom. All swimming sessions were performed between 12:00 and 18:00 hours.

At the end of swimming sessions, rats were withdrawn from the cylinders, dried with towels, placed in hot boxes for 15 minutes for resting and recovery, and then put back to their housing boxes.

Two sessions were performed: an initial pre-test for 15 minutes followed by a 5-minute test, after 16 days. The drugs were given between both sessions.

Each animal was randomly assigned to one treatment and used for one single session of pre-test/test.

Behavioral Scoring

Rats were studied at 5-second intervals during the session of forced swimming session. At each interval of 5 seconds, the predominant behavior was assigned to one of the following categories: (1) immobility: flotation in water without fighting, only doing the necessary movements to keep the head out of the water; (2) swimming, making the movements of active swimming, plus the ones necessary to keep simply the head out of the water (i.e., moving around the cylinder); and (3) climbing, making active movements with fore hints out of the water, usually against walls. The scoring for each behavior was expressed as the total of records per session of 5 minutes.

The method of behavioral sampling distinguishes classes of antidepressant drugs: for example, NRIs reduce the immobility and increase the climbing without affecting the swimming, while the SSRIs reduce the immobility and increase the swimming without changes in the climbing (Heisler L K, Kanarek R B., Gerstein A. Fluoxetine Decreases Fat and Protein Intakes But Not Carbohydrate Intake in Male Rats. Pharmacology Biochemistry and Behavior, Volume 58, Issue 3, November 1997, pp 767-773).

Open Field Test

This test was used to determine if treatments which were effective in FST studies showed unspecific effects on the locomotor activity in rats previously exposed to forced swimming.

These studies were performed exactly the same way as the studies of FST had been carried.

All rats that experimented the first day of FST (pre-test), received the different treatments during 16 days. On day 17 they were placed in the cage of open field (60×60×60 cm) with the floor divided in squares (15×15 cm). The tests were performed between 14:00 and 17:00 hours, illuminated with an electric bulb of 75 W and placed 75 cm above the floor of the cage, in a quiet room.

During all the experiments the room was dark. All the animals were placed carefully in the middle of the square of the open field which they explored freely.

The locomotion was measured by the number of squares they enter with the four legs (counts), over a period of 5 minutes. After withdrawing the animal, the open field was carefully cleaned with a damp wipe.

The behavior was recorded by an observer who ignored the test treatments previously performed in the animals and the results were expressed as the mean±standard error.

Analysis of Phospholipids

After the experiments were ended, the animals were euthanized by inhalation of CO₂ and beheaded. Fresh desiccated hemispheres were used for the analysis of lipids. The brain cortex was removed, followed by the hippocampus. All regions were frozen with dry ice and samples were stored at −80° C. until the moment of analysis.

The lipids were extracted by the method of Bligh and Dryer (Bligh, E G and Dyer, W J. A rapid method of total lipid extraction and purification. Can. J. Biochem. Physiol. T37T (1959), pp. 911-913). The phospholipids were separated by one-dimensional thin layer chromatography of two solvents (TLC) in Silica gel G plates (0.25 mm of thickness). The first system of solvent used was a mixture of chloroform/methanol/acetic acid/water (120:30:30:3 v/v/v/v), and the second one was, chloroform/methanol/acetic acid/water (120:46:19:3 v/v/v/v). The lipids were detected with vapors of I₂. Spots on the TLC plates corresponding to each species of phospholipids were removed, and the phospholipids were quantified by the method of Fiske and Subbarrow (Fiske, L M and Subbarow, Y. J. Biol. Chem. T66T (1925), pp. 375-389).

Statistical Analysis

For each experiment, one- or two-way analysis of variance (ANOVA) were done with the antidepressant treatment and the diet as factors. The subsequent post-hoc analysis were performed with Dunnett's or Tukey's comparisons. Statistically significant differences were considered if P<0.05.

1. A method for the treatment of psychiatric disorders, characterized for comprising administering to a subject in need thereof an amount of fatty acids and a suboptimal dose of at least one antidepressant.

2. The method according to claim 1, characterized for the fact that the fatty acid is omega-3 (ω3) and is selected from the group comprising of docohexaenoic acid (DHA) and eicosapentaenoic acid (EPA).

3. The method according to claim 1, characterized for the fact that the antidepressant is selected from the group comprising of fluoxetine and mirtazapine.

4. The method according to claim 1, characterized for the fact that the sub-optimal dose of the antidepressant is a dose not producing side effects.

5. The method according to claim 1, characterized for the fact that the sub-optimal dose of the antidepressant is between ½ and 1/100 of the optimal dose.

6. The method according to claim 1, characterized for the fact that the sub-optimal dose of the antidepressant is a dose lower than 5 mg/kg.

7. The method according to claim 1, characterized for the fact that the sub-optimal dose of the antidepressant is a dose lower than 3 mg/kg.

8. The method according to claim 1, characterized for the fact that the fatty acid is administered orally.

9. The method according to claim 1, characterized for the fact that the antidepressant is administered systemically.

10. The method according to claim 1, characterized for the fact that the antidepressant is administered orally.

11. The method according to claim 1, characterized for the fact that the psychiatric disorder is selected from the group comprising of major depression, bipolar depression, unipolar depression and depression resistant to pharmacological treatment.

12. The method according to claim 8, characterized for the fact that the fatty acid is administered orally at a dose between 0.15 and 1.00 g/kg/day.

13. A method to reduce body weight of an individual, characterized for comprising administering to a subject in need thereof an amount of fatty acids and a suboptimal dose of at least one antidepressant.

14. The method according to claim 13, characterized for the fact that the fatty acid is omega-3 (ω3) and is selected from the group comprising of docohexaenoic acid (DHA) and eicosapentaenoic acid (EPA).

15. The method according to claim 13, characterized for the fact that the antidepressant is mirtazapine.

16. The method according to claim 13, characterized for the fact that the sub-optimal dose of the antidepressant is a dose not producing side effects.

17. The method according to claim 13, characterized for the fact that the sub-optimal dose of the antidepressant is between ½ and 1/100 of the optimal dose.

18. The method according to claim 13, characterized for the fact that the sub-optimal dose of the antidepressant is a dose lower than 5 mg/kg.

19. The method according to claim 13, characterized for the fact that the sub-optimal dose of the antidepressant is a dose lower than 3 mg/kg.

20. The method according to claim 13, characterized for the fact that the fatty acid is administered orally.

21. The method according to claim 13, characterized for the fact that the antidepressant is administered systemically.

22. The method according to claim 13, characterized for the fact that the antidepressant is administered orally.

23. The method according to claim 14, characterized for the fact that the fatty acid is administered orally at a dose between 0.15 and 1.00 g/kg/day.
